(19) United States
(12) Patent Application Publication
(10) Pub. No.: US 2023/0285342 A1
(43) Pub. Date: Sep. 14, 2023

(54) COMPOSITIONS AND METHODS

(71) Applicant: Spectrix Therapeutics, LLC, Northlake, TX (US)

(72) Inventor: Mark Tengler, Northlake, TX (US)

(21) Appl. No.: 18/302,899

(22) Filed: Apr. 19, 2023

Related U.S. Application Data
(63) Continuation-in-part of application No. 17/743,337, filed on May 12, 2022.
Application No. 17/743,337 is a continuation-in-part of application No. 15/808,494, filed on Nov. 9, 2017.
Application No. 15/808,494 is a continuation-in-part of application No. 15/583,695, filed on May 1, 2017, now abandoned.
(60) Provisional application No. 63/187,511, filed on May 12, 2021.
(60) Provisional application No. 62/331,148, filed on May 3, 2016.
(60) Provisional application No. 62/334,271, filed on May 10, 2016.

(51) Int. Cl. A61K 31/198; A61K 9/20

(52) U.S. Cl. CPC A61K 31/198; A61K 9/2081; A61K 9/2095

(57) ABSTRACT

The present invention includes a compositions and methods comprising: one or more thyroid hormones in or on a micro-multi-particulate having a mean particle size of 150 uM or smaller and a Span (D90−D10)/(D50) of less than 2.0, wherein the total thyroid hormone(s) are less than 10% weight-to-weight (w/w) of the micro-multi-particulate; and a polymer release coating on the micro-multi-particulate that is greater than a 5:1 ratio w/w, on a dry weight basis, to a dry weight of the thyroid hormone(s), wherein a total tablet weight exceeds a micro-multi-particulate weight by a ratio of 5:1 or greater.

### CROSS-REFERENCE TO RELATED APPLICATIONS

This application is a continuation-in-part application of U.S. patent application Ser. No. 17/743,337 filed on May 12, 2022, which claims priority based on U.S. provisional Application No. 63/187,511, filed May 12, 2021; and is a continuation-in-part application of U.S. patent application Ser. No. 15/808,494 filed on Nov. 9, 2017, which is a continuation-in-part of U.S. patent application Ser. No. 15/583,695 filed on May 1, 2017, which claims priority based on U.S. Provisional Application No. 62/331,148 filed on May 3, 2016 and 62/344,271 filed on Jun. 1, 2016, the contents of each are incorporated by reference in their entirety.

### TECHNICAL FIELD OF THE INVENTION

The present inventions relate in general to the of field of composition and methods for high potency drug delivery in a micro-multi-particulate extended-release chewable tablet.

### STATEMENT OF FEDERALLY FUNDED RESEARCH

None.

### BACKGROUND OF THE INVENTION

Without limiting the scope of the invention, its background is described in connection with treatments for nanoparticulate compositions.

U.S. Pat. No. 9,220,788, issued to Davis, et al., is entitled “Nanoparticle and polymer formulations for thyroid hormone analogs, antagonists, and formulations and uses thereof.” Briefly, the invention is said to include methods of treating subjects having conditions related to angiogenesis including administering an effective amount of a polymeric nanoparticle form of thyroid hormone agonist, partial agonist or an antagonist thereof, and to promote or inhibit angiogenesis in the subject.

U.S. Pat. No. 7,723,390, issued to Garavani, is entitled, “Pharmaceutical formulations for thyroid hormones”. Briefly, the invention is said to provide for pharmaceutical formulations based on thyroid hormones enabling a safe and stable oral administration in the framework of the strict therapeutic index prescribed in case of thyroid disorders.

United States Patent Publication No. 20070099841, filed by Moncrief, et al., is entitled “Prodrugs of T3 and T4 with enhanced bioavailability”. These applicants are said to teach compositions of amino acid and peptide conjugates comprising T3 and/or T4. The T3 or T4 is covalently attached to at least one amino acid via the N-terminus, the C-terminus, a side chain of the peptide carrier, and/or interspersed within the peptide chain. Also discussed are methods for protecting and administering active agents and methods for treating thyroid disorders.

Despite these efforts, a need remains for novel compositions and methods for the controlled delivery of active agents, and more particularly, for the delivery of highly potent active agents, i.e., those compounds in which a therapeutic dose is in micrograms versus milligrams or grams.

### SUMMARY OF THE INVENTION

In one embodiment, the present invention includes a tablet comprising: one or more thyroid hormones in or on a micro-multi-particulate having a mean particle size of 150 uM or smaller and a Span (D90−D10)/(D50) of less than 2.0, wherein the total thyroid hormone(s) are less than 10% weight-to-weight (w/w) of the micro-multi-particulate; and a polymer release coating on the micro-multi-particulate that is greater than a 5:1 ratio w/w, on a dry weight basis, to a dry weight of the thyroid hormone(s), wherein a total tablet weight exceeds a micro-multi-particulate weight by a ratio of 5:1 or greater. In one aspect, a dosage form is an extended-release chewable tablet. In another aspect, the tablet is flavored. In another aspect, the tablet has a non-gritty mouth feel. In another aspect, the micro-multi-particulate comprises a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In another embodiment, the present invention includes a composition comprising: a micro-multi-particulate having a mean particle size of less than 150 uM and a Span (D90−D10)/(D50) of less than 2.0, wherein the micro-multi-particulate are loaded with one or more thyroid hormones and the total thyroid hormone(s) are less than 10% w/w of a total micro-multi-particulate weight; and a polymer release coating on the micro-multi-particulate that is greater than a 5:1 ratio w/w, on a dry weight basis, to a dry weight of total thyroid hormones; wherein a total composition weight exceeds the micro-multi-particulate by a ratio of 5:1 or greater and is flavored. In one aspect, a dosage form is an extended-release chewable tablet. In another aspect, the tablet is flavored. In another aspect, the tablet has a non-gritty mouth feel. In another aspect, the micro-multi-particulate comprises a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In another embodiment, the present invention includes an extended release chewable tablet comprising: a liothyronine (T3) in or on a micro-multi-particulate having a mean particle size of less than 150 uM and a Span (D90−D10)/(D50) of less than 2.0, wherein the T3 is less than 10% w/w of a total micro-multi-particulate weight after loading; and a polymer release coating on the micro-multi-particulate that is greater than a 5:1 ratio w/w, on dry weight basis to a dry weight of T3; wherein a total tablet weight of the tablet exceeds the micro-multi-particulate weight by a ratio of 5:1 or greater, and the chewable tablet is flavored. In another aspect, a therapeutic performance of a T3 dosage meets or exceeds FDA standards for hypothyroidism, selected from at least one of: content uniformity, assay, dissolution, or stability. In another aspect, the tablet is flavored. In another aspect, the tablet has a non-gritty mouth feel. In another aspect, the micro-multi-particulate comprises a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the T3 is 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In one embodiment, the present invention includes a method of making a pharmaceutical composition comprising: contacting one or more thyroid hormones to a micro-multi-particulate with a mean particle size of 150 uM or smaller and a Span (D90−D10)/(D50) of less than 2.0, wherein the total weight of the thyroid hormones is less than 10% of the micro-multi-particulate weight; and coating a polymer release coating on the micro-multi-particulate that is greater than a 5:1 ratio w/w, on a dry weight basis, to a dry weight of the thyroid hormone(s), wherein a total tablet weight of the composition exceeds a micro-multi-particulate weight by a ratio of 5:1 or greater. In one aspect, a dosage form is an extended-release chewable tablet. In another aspect, the tablet is flavored. In another aspect, the tablet has a non-gritty mouth feel. In another aspect, the micro-multi-particulate comprises a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In another embodiment, the present invention includes a method of treating hypothyroidism, the method comprising: administering to a subject in need thereof a composition comprising one or more thyroid hormones in or on a micro-multi-particulate having a mean particle size of 150 uM or smaller and a Span (D90−D10)/(D50) of less than 2.0, wherein the total thyroid hormone(s) are less than 10% weight-to-weight (w/w) of the micro-multi-particulate, and a polymer release coating on the micro-multi-particulate that is greater than a 5:1 ratio w/w, on a dry weight basis, to a dry weight of the thyroid hormone(s), wherein a total tablet weight of the composition exceeds a micro-multi-particulate weight by a ratio of 5:1 or greater, in an amount sufficient to treat the hypothyroidism. In one aspect, a dosage form of the composition is an extended-release chewable tablet. In another aspect, the tablet is flavored. In another aspect, the tablet has a non-gritty mouth feel. In another aspect, the micro-multi-particulate comprises a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In another embodiment, the present invention includes a multi-particulate tablet comprising: a micro-multi-particulate comprising one or more thyroid hormones, wherein the micro-multi-particulate has a mean particle size of 150 uM or smaller and a Span (D90−D10)/(D50) of less than 2.0, wherein the total thyroid hormone(s) is less than 10% weight-to-weight (w/w) of the micro-multi-particulate; and a polymer release coating on the micro-multi-particulate that is greater than a 5:1 ratio w/w, on a dry weight basis, to a dry weight of the thyroid hormone(s), wherein a total tablet weight exceeds a micro-multi-particulate weight by a ratio of 5:1 or greater. In one aspect, a dosage form of the tablet or composition is an extended-release chewable tablet. In another aspect, the tablet is flavored. In another aspect, the tablet has a non-gritty mouth feel. In another aspect, the micro-multi-particulate comprises a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the one or more thyroid hormones are T3, T4, or both. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the FloVitro results comparing the dissolution concentration over time of Synthroid, levothyroxine (T4) on an IRP64 resinate, and levothyroxine (T4) on a coated IRP64 resinate following the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention can be used for the therapeutic delivery of one or more thyroid hormones in an extended-release chewable tablet. Although, hypothyroidism is the most prescribed indication in the United States, no product exists that can deliver therapeutic performance that meets FDA standards, including at least one of: content uniformity, assay, dissolution, and/or stability for thyroid hormones.

Thyroid hormones are prescribed in microgram quantities making the drug delivery formulation extremely difficult. Furthermore, the extended release of these hormones has never been achieved in an approved pharmaceutical product.

In one embodiment of the invention, the thyroid hormone is dissolved to achieve phase transition and complete uniformity prior to being complexed with the micro-multi-particulate substrate at a potency of about 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% weight-to-weight of drug on a micro-multi-particulate. This Dynamic Multiphase Magnitudinous (DMM) dilution to reduce potency is part of the present invention. These low levels of drug loading to substrate are unique, and is the opposite of the prior art, which teaches how to maximize drug to substrate ratios and therefore teaches away from DMM. A Dynamic Multiphase Magnitudinous dilution is a process of dissolving a solid active to form a liquid solution and then allowing a phase change back to as solid to form on a homogeneous solid, wherein the phase change dilution is equal or greater than an order of magnitude, e.g., 10:1, 100:1, 1,000:1, 10,000:1 or greater.

Non-limiting examples of micro-multi-particulate substrates for use with the present invention include, e.g., polyethylene glycol (PEG)(known generally by their average molecular weight, e.g., PEG1000, PEG2000, PEG3000, PEG4000, PEG5000, and higher), resins, ion exchange resins, lactose, mannitol, lipids, micro-crystalline cellulose, or any pharmaceutically acceptable particulate, blend or composition of particulates that are able to be sized with a mean particle size of less than 150 microns and a span of less than 2.0 and subsequently coated.

Next, a portion of, or all of, the pre-sized and loaded micro-multi-particulates (MMPs) are coated to impart an extended-release profile on all or a portion of the population of MMPs wherein the polymer release coating of MMP exceeds the thyroid hormone by a 1:1, 5:1, 10:1, 11:1, 12:1, 13:1, 14:1, 15:1, 16:1, 17:1, 18:1, or 20:1 ratio (w/w) on a dry weight basis. In certain embodiments the micro-multi-particulates are for immediate release and not coated. In certain embodiments the micro-multi-particulates are for immediate release and coated.

Finally, the uncoated and/or coated MMPs that have undergone DMM are further wet granulated with tableting excipients and it is this DMM/MMP-granulate that is blended with additional tableting excipients to form a tablet, wherein the total tablet weight exceeds the DMM/MMP weight by 1:1, 5:1, 10:1, 11:1, 12:1, 13:1, 14:1, 15:1, 16:1, 17:1, 18:1, or 20:1. In certain embodiments, the tablet is flavored and/or has a non-gritty mouth feel when chewed.

In certain embodiments, the active agent, such as thyroid hormone(s), are loaded onto the micro-multi-particulate substrate at a temperature in which the active agent is not degraded, such as 25, 30, 35, 40, 45, 50, 60, or 65 degrees Celsius. Avoiding a temperature at which the active agent is degraded is critical for, e.g., highly potent, heat-labile active agents such as thyroid hormone(s), which have a strict clinical dosage level and for which very small variations are not acceptable. Any degradation of these highly potent active agents, e.g., 1, 2, 3, 4, 5, 6, 7, 8, 9, or 10% degradation of the active agent causes a significant deviation from FDA approved degradation and causes the rejection of the formulation. Elimination or significant reduction of any degradation is critical for agents such as thyroid hormone(s), in particular T3 and T4, which are known to degrade at higher temperatures. The present invention loads the active agent at a temperature that does not induce degradation, such that the degradation is less that 0.1, 0.2, 0.3, 0.4, 0.5, 0.6, 0.7, 0.8, 0.9, 1, 2, 3, 4, 5, 6, 7, 8, 9, or 10% of the initial concentration of active agent.

When dosing highly potent, heat-labile active agents such thyroid hormone(s), preventing uncontrolled release upon exposure to water is another important parameter. For example, the prior art used humectants and/or hyper-loading of active agent (24% or greater active agent weight-to-weight of an ion-exchange resin) at high temperature. The high temperature is necessary to swell the resin to allow for the hyper-loading of the active agent, such that when the temperature is lowered the active agent fills the spaces to prevent rupture of a coating.

The present invention provides stable (potency remains within 95%-105% of label claim), coated particles that are loaded at a temperature in which the active agent is not degraded, i.e., below 65 degrees Fahrenheit and less than 10%, 9%, 8%, 7%, 6%, 5%, 4%, 3%, 2%, 1% degradation and without the need for humectants or hyper-loading of the active agent on the ion-exchange resin. In fact, the present invention overcomes these problems with the prior art by doing the exact opposite of the prior art, namely, dosing the active agent at a very low weight-percent on the micro-multi-particulate substrate to provide for highly controlled loading, that is, very precise, i.e., having a blend uniformity had a relative standard of deviation of 2% or less for 3 or more replicate samples, weight percent loading to meet the strict dosing requirements from the FDA (i.e., USP <905> uniformity of dosage units). In one example, the one or more thyroid hormones are 0.02, 0.04, 0.06, 0.08, 0.09, 0.1, 0.2, 0.3, 0.4, 0.5, 0.6, 0.8, 0.9, 1, 2, 3, 4, 5, 6, 7, 8, 9 or 10% weight-to-weight (w/w) of the total microparticulate weight.

Further, the present invention also includes DMM dilution and loading of thyroid hormones onto the micro-multi-particulate substrate, followed by a solid-to-solid geometric dilution (i.e., equal parts blended together to aid in uniformity, may be repeated to increase dilution factor). Once loaded, the level of active agent loaded onto the micro-multi-particulate substrate can be determined, and then further geometrically diluted to the final dosage. In this embodiment, the loaded micro-multi-particulate substrate having a known level of loading is then measured and formulated with excipients into a final dose. The tablets can include indicia to allow for visual identification of tablets with different dosages such as color, shape, size, imprints, etc., that will identify the dosage of the active agents in the final tablet.

As used herein, the term “pharmaceutically effective amount” refers to that amount of an agent effective to produce the intended effect of reducing, and/or preventing hypothyroidism.

Hypothyroidism may be caused by decreased production of thyroid hormones. Such factors include loss of thyroid tissue due to disease or surgery.

Pharmaceutical composition refers to a composition suitable for pharmaceutical use in an animal or animal cell line. The animal may be a mammal, such as a human. A pharmaceutical composition of the invention includes a pharmaceutically effective amount of one or more thyroid hormones or analogs thereof, and optionally a pharmaceutically acceptable resin.

As used herein, the term “active agent” refers to any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. In certain examples, a hyper-potent active is an active agent dosed at 1 mg per dose, or less than 1 mg per dose, such as 0.1, 0.5, 1, 5, 10, 15, 20, 25, 30, 35, 40, 45, 50, 60, 70, 75, 80, 90, 100, 150, 200, 225, 250, 275, 300, 400, 450, 500, 550, 600, 650, 700, 750, 800, 850, 900, or 950 micrograms.

As used herein, the term “extended release” means a dosage form that allows at least about a two-fold reduction in dosage frequency as compared to that drug presented as an immediate-release (conventional) dosage form. Examples of extended-release dosage forms include controlled-release, sustained-release, and long-acting drug products.

As used herein, the term “immediate release” refers to a micro-multi-particulate composition that release at least 80% of the active agent within 1 hour.

As used herein, the term “microparticle” refers to a drug formulation in discrete particulate form, and is interchangeable with the terms: “microspheres”, “spherical particles”, “microcapsules”, “particles”, “multiparticulates”, “granules”, “spheroids”, beads”, spherules and “pellets”.

As used herein, the term “micro-multi-particulate” refers to a microparticle within a defined size range, namely, a mean particle size of 150 micrometer (uM) or smaller and a Span (D90-D10)/(D50) of less than 2.0. Non-limiting examples of micro-multi-particulate comprise ion exchange resin, PEG, lactose, mannitol, microcrystalline cellulose, sorbitol, carnauba wax, and optionally, other pharmaceutically acceptable carriers.

As used herein, the term “modified release” refers to drug products that alter the timing and/or the rate of release of the drug substance (e.g., thyroid hormone or analog). A modified-release dosage form is a formulation in which the drug-release characteristics of time course and/or location are chosen to accomplish therapeutic, or convenience objectives not offered by conventional dosage forms such as solutions, ointments, or promptly dissolving dosage forms. Several types of modified-release oral drug products are recognized. Non-limiting examples include delayed release and extended-release dosage forms.

The terms “reduced”, “reduction” or “decrease”, “down-regulate” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

As used herein, the term “flavorant” is intended to mean a compound used to impart a pleasant flavor and often odor to a pharmaceutical preparation. In addition to the natural flavorants, many synthetic flavorants are also used. Such compounds include, by way of example and without limitation, anise oil, cinnamon oil, cocoa, menthol, orange oil, peppermint oil and vanillin and the like.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and the like.

As used herein, the term “tablet antiadherents” is intended to mean agents which prevent the sticking of table formulation ingredients to punches and dies in a tableting machine during production. Such compounds include, by way of example and without limitation, magnesium stearate, talc, and the like.

As used herein, the term “tablet binders” is intended to mean substances used to cause adhesion of powder particles in table granulations. Such compounds include, by way of example and without limitation, acacia, alginic acid, carboxymethyl cellulose, sodium, compressible sugar ethylcellulose, gelatin, liquid glucose, methylcellulose, povidone and pregelatinized starch and the like.

As used herein, the term “tablet and capsule diluent” is intended to mean inert substances used as fillers to create the desired bulk, flow properties, and compression characteristics in the preparation of tablets and capsules. Such compounds include, by way of example and without limitation, dibasic calcium phosphate, kaolin, lactose, mannitol, microcrystalline cellulose, powdered cellulose, precipitated calcium carbonate, sorbitol, and starch and the like.

As used herein, the term “tablet direct compression excipient” is intended to mean a compound used in direct compression tablet formulations. Such compounds include, by way of example and without limitation, dibasic calcium phosphate and the like.

As used herein, the term “tablet disintegrant” is intended to mean a compound used in solid dosage forms to promote the disruption of the solid mass into smaller particles that are more readily dispersed or dissolved. Such compounds include, by way of example and without limitation, alginic acid, carboxymethylcellulose, calcium, microcrystalline cellulose, polacrilin potassium, sodium alginate, sodium starch glycolate, and starch and the like.

As used herein, the term “tablet glidant” is intended to mean agents used in tablet and capsule formulations to reduce friction during tablet compression. Such compounds include, by way of example and without limitation, colloidal silica, cornstarch, talc, and the like.

As used herein, the term “tablet lubricant” is intended to mean substances used in tablet formulations to reduce friction during tablet compression. Such compounds include, by way of example and without limitation, calcium stearate, magnesium stearate, mineral oil, stearic acid, zinc stearate, and the like.

As used herein, the term “tablet/capsule opaquant” is intended to mean a compound used to render a capsule or a tablet coating opaque. An opaquant may be used alone or in combination with a colorant. Such compounds include, by way of example and without limitation, titanium dioxide and the like.

As used herein, the term “tablet polishing agent” is intended to mean a compound used to impart an attractive sheen to coated tablets. Such compounds include, by way of example and without limitation, carnauba wax, white wax, and the like.

As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

It should be understood that compounds used in the art of pharmaceutical formulation generally serve a variety of functions or purposes. Thus, if a compound named herein is mentioned only once or is used to define more than one term herein, its purpose or function should not be construed as being limited solely to that (those) named purpose(s) or function(s).

For oral therapeutic administration, the micro-multi particulate containing the active compound(s) may be incorporated with excipients and used in the form of ingestible tablets, buccal tables, troches, capsules, elixirs, suspensions, syrups, wafers, and the like. Such compositions and preparations should contain at least the minimal therapeutic amount per dose. The percentage of the compositions and preparations may, of course, be varied and may conveniently be between about 0.001% to about 80% of the weight of the unit. The amount of particles containing the active compound(s) in such therapeutically useful compositions is such that a suitable dosage will be obtained.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference, and the like, relevant portions incorporated herein by reference.

For example, the one or more thyroid hormones may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The thyroid hormone(s) or analogs thereof may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the thyroid hormone(s) or analogs thereof may be coupled one or more biodegradable polymers to achieve controlled release of the thyroid hormone(s) or analogs thereof, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the thyroid hormone(s) or analogs thereof and powdered carriers, such as lactose, starch, cellulose derivatives, magnesium stearate, stearic acid, and the like. Like diluents may be used to make compressed tablets. Both tablets and capsules may be manufactured as immediate-release, mixed-release or modified-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract. Furthermore, these properties can be imparted directly on the particles themselves to achieve the same effect.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Anti-oxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl-or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

Capsules. Capsules may be prepared by filling standard two-piece hard gelatin capsules each with 10 to 500 milligrams of particles containing active ingredient.

Soft Gelatin Capsules. Active particles are suspended in a digestible oil such as soybean oil, cottonseed oil or olive oil. The active particles are prepared and injected by using a positive displacement pump into gelatin to form soft gelatin capsules containing, e.g., 10-500 micrograms of the active thyroid hormone. The capsules are washed and dried.

Tablets. A large number of tablets are prepared by conventional procedures so that the dosage unit was 0.1, 0.5, 1, 2, 3, 4, 5, 6, 7, 8, 9, 10, 15, 20, 25, 30, 40, 50, 60, 75, 70, 80, 90, 100, 110, 120, 125, 130, 140, 150, 160, 175, 180, 190, 200, 210, 220, 225, 230, 240, 250, 260, 270, 275, 280, 290, 300, 325, 350, 375, 400, 425, 450, 475, or 500 micrograms of active thyroid hormone, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the thyroid hormone(s) particles or analogs thereof, drug and/or salt thereof, conventional beading or filling agents and, optionally, sweeteners, flavors and lubricants.

For mini-tablets, the active thyroid hormone particles are compressed into a tablet with a hardness in the range 0.5 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

The present invention also includes pharmaceutical kits useful, for example, for the treatment of hypothyroidism, which comprise one or more containers containing a pharmaceutical composition comprising a therapeutically effective amount of the one or more thyroid hormones. Such kits may further include, if desired, one or more of various conventional pharmaceutical kit components, such as, for example, tablets comprising the micro-multi-particulate with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

In one example, the present invention includes a pharmaceutical composition comprising one or more thyroid hormones or analogs thereof, wherein the first thyroid hormone is formulated for immediate release and the second thyroid hormone is formulated for modified release. For example, the one or more of the thyroid hormones are bound to a microparticle, such as an ion exchange resin. Non-limiting examples of the one or more thyroid hormones for use with the present invention can be selected from T4, T3, T4 or T3 N-Methyl, T4 or T3 N-Ethyl, T4 or T3 N-Triphenyl, T4 or T3 N-Propyl, T4 or T3 N-Isopropyl, T4 or T3-N-Tertiary butyl, GC-1, DIPTA, Tetrac and Triac. The two or more thyroid hormones are provided in an amount effective to treat hypothyroidism.

In addition to the two or more thyroid hormones, the composition of the present invention may further comprise one or more biologically active substances that help potentiate the activity of the thyroid hormone(s)s or analogs thereof. Generally, the composition will be adapted for the treatment of hypothyroidism by providing the most common dosage amounts for the equivalent hormone(s).

In one specific embodiment, the two or more thyroid hormones are T4 and/or T3 attached to an ion exchange resin that prevents polymorphism in the crystalline structure. In another example, binding the thyroid hormone to resin provides a geometric dilution to aid in the ease of manufacturing and increase consistency in dosing. Often, the modified release thyroid hormone is T3. The composition of the present invention can be formulated as a liquid suspension, chewable composition, orally disintegrating tablet, or a swallowed tablet composition.

In another specific example, the two or more thyroid hormones are T4 and T3, and are provided a ratio of T4:T3 is from 1:1, 5:1, 10:1, 11:1, 12:1, 13:1, 14:1, 15:1, 16:1, 17:1, 18:1, or 20:1. These hormones can be provided as a modified release orally disintegrating tablet. For example, the T4, T3, and/or analogs thereof, can be attached to a microparticle, e.g., ion-exchange resin particles that are acidic cation exchange resins. For example, the ion-exchange resin particles can be basic anion exchange resin. The resin may be further coated, e.g., coating of the one or more modified release drug resin particles comprises a triggered-release coating that is triggered by a pH change. Certain non-limiting examples of coatings for use with the present invention include, e.g., cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters, co-polymerized methacrylic acid/acrylic acid ethyl esters, or mixtures thereof. The modified release coating can also be a non-pH dependent controlled release coating.

The dosages of the present invention can vary to meet the needs of an individual user, or can be produced in large batches having specific amounts of the one or more thyroid hormones or equivalents thereof based on the most commonly used amounts. For example, the amount of the one or more thyroid hormones can be from 0.013 to 0.30 mg equivalent of levothyroxine sodium per dose.

The ionic exchange resin and coating can be selected such that greater than 40% of the first thyroid hormone is released within the first 45 minutes after the product is introduced into an in vitro dissolution assay, wherein the conditions of the dissolution assay are an initial dissolution medium of 0.1 N HCL, and after 2 hours, the medium is adjusted to a pH of about 6.8; and the dissolution assay is performed using a USP Apparatus 2.

Another example of the present invention includes a pharmaceutical composition comprising thyroid hormone complexed with ion-exchange resin particles to form drug resin particles, wherein the composition comprises a first plurality of immediate release drug-resin particles and a second plurality of drug-resin particles that are coated for modified release coating, wherein the composition has an in vivo fasted serum profile with a first and second peak wherein the first peak occurs before 3 hours after ingestion of the composition and the second peak occurs after 3 hours after ingestion.

Another example of the present invention includes a method of making a pharmaceutical composition comprising, consisting essentially, of, or consisting of, attaching one or more thyroid hormones or analog thereof with ion-exchange resin particles to form drug-resin particles, wherein at least 30% by weight of the first thyroid hormone or more is formulated for immediate release; and a second thyroid hormone is formulated for modified release.

Another example of the present invention includes a method of evaluating a composition of the present invention believed to be useful in treating hypothyroidism, the method comprising: a) measuring the blood levels of one or more thyroid hormone(s) from a first set of subjects suspected of having hypothyroidism; b) administering the composition to a first subset of the patients, and a placebo to a second subset of the patients; c) repeating step a) after the administration of the composition or the placebo; and d) determining if the composition reduces the number of hypothyroidism symptoms or endpoints that are statistically significant or non-inferior as compared to any reduction occurring in the second subset of patients, wherein a statistically significant reduction indicates that the composition is useful in treating hypothyroidism and non-inferior indicates that it is no less effective than the comparator.

The compositions disclosed herein are useful in the treatment of disease or disorders mediated by thyroid hormones.

In a particular embodiment, a method is provided for treating diminished or absent thyroid function (e.g., hypothyroidism) in a subject in need thereof, thereby treating the diminished or absent thyroid function. The diminished or absent thyroid function may result from functional deficiency, primary atrophy, partial or complete absence of the thyroid gland, or the effects of surgery, radiation, or antithyroid agents.

In certain embodiments, the method of treatment produces a reduction in one or more symptoms selected from fatigue, weight gain, dry skin and cold intolerance, muscle weakness and cramps, memory impairment, depression, constipation and neurocognitive deficits.

In certain embodiments, the subject in need thereof has been diagnosed with overt hypothyroidism.

In other embodiments, the subject in need thereof has been diagnosed with subclinical hypothyroidism.

Table 1 is a comparative example showing the release profiles for Synthroid, an uncoated resinate, and the coated micro-multi-particulate of the present invention.

| | Cmax | | Tmax | | |
|---|---|---|---|---|---|
| Sample | (mg/L) | RSD | (min) | RSD | Notes |
| Synthroid tablets | 0.136 | — | 148 | — | Single Run |
| Coated IRP64 | 0.097 | — | 716.5 | — | Single Run |
| Resinate | | | | | |
| Uncoated IRP64 | 0.100 | — | 140 | — | Single Run |
| Resinate | | | | | |

Coated Resinate mass = 215.0 mg
Uncoated Resinate mass = 174.1 mg

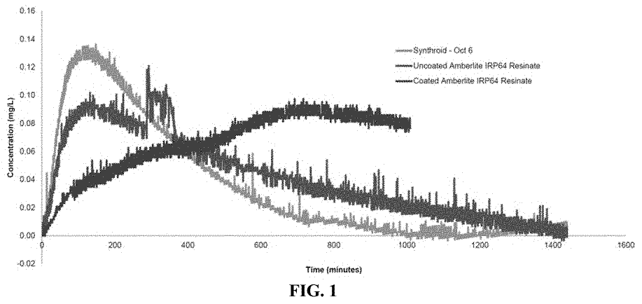

FIG. 1 is a graph that shows the FloVitro results comparing the dissolution concentration over time of Synthroid, levothyroxine (T4) on an IRP64 resinate, and levothyroxine (T4) on a coated IRP64 resinate following the present invention.

TABLE 2
Loading and Coating Steps, T4 and T3

| | Sizing of microparticle | | | |
|---|---|---|---|---|
| Step 1 | Item | grams | Size exclusion sieving | Yield grams |
| | IRP-64 resinate | 250000 | | 11500 |
| | Drug loading of sized Microparticle | | | |
| Step 2 | Item | grams | | |
| | Sized IRP-64 | 11500 | | |
| | Levothyroxine Sodium | 116.667 | | |
| | Liothyronine Sodium | 8.333 | | |
| | NaOH | 68 | Removed in processing | |
| | USP purified water | 101000 | Removed in processing | |
| | | | Dry weight | 11625 |
| | Making of XR-coating solution | | | |
| Step 3 | Item | grams | | |
| | Ethyl alcohol | 19440 | | |
| | USP purified water | 2160 | | |
| | Triethylcitrate | 176 | | |
| | Ethylcellulose | 2000 | | |
| | Hydroxypropyl Cellulose | 224 | | |
| | | | Dry weight | 24000 |
| | Coating of Microparticle | | | |
| Step 4 | Item | grams | | |
| | Drug loaded sized particle | 12000 | | |
| | XR-Coating solution exceeding | | | |
| | 5:1 (coating solids:drug) | 6250-24000 | | |
| | | | Total | 33600 |
| | | | Dry weight | 14160 |
| | Wet granulation of drug loaded uncoated | | | |
| | and coated microparticles | | | |
| Step 5 | Item | grams | | |
| | IR-drug loaded uncoated MP | 1060 | | |
| | XR-drug loaded coated MP | 7085 | | |
| | Mannitol | 28360 | | |
| | Microcrystalline cellulose | 9750 | | |
| | Povidone | 3750 | | |
| | USP Purified water | 13450 | Removed in processing | |
| | | | Total | 63455 |
| | | | Dry weight | 50005 |
| | Blending of granulation with tableting excipients | | | |
| Step 6 | Item | grams | | |
| | IR/XR granulation | 50000 | | |
| | Microcrystalline cellulose | 31180 | | |
| | Mannitol | 92200 | | |
| | Avicel CE15 | 44800 | | |
| | Flavor | 2240 | | |
| | lake Dye | 224 | | |
| | Mag Stearate | 3360 | | |
| | | | Total | 224004 |

TABLE 3
Basic Parameters

| Step 1 | Critical parameters | | | Notes |
|---|---|---|---|---|
| | Particle size | | | Fraction collected |
| | 80 mesh-270 | | | retained on 270 mesh |
| | mesh | | | |
| Step 2 | pH | Temp | Time | lower temp loading |
| | .02N NaOH | <60 F. | <5 hrs | for short time under |
| | solution | | | basic conditions |
| Step 3 | solubility | mixing time | | |
| | complete | >10 min | | |
| Step 4 | agglomeration | | | fluidized and sprayed |
| | <10% | | | to prevent |
| | | | | agglomeration |
| Step 5 | time | RPM | | |
| | 1-25 min | 10-500 | | |
| Step 6 | Revolutions | | | |
| | 10-200 | | | |

TABLE 4
Other parameters.

| | Ranges | | Substitutable ingredients |
|---|---|---|---|
| | Min | Max | micro particle |
| Step 1 | 400 mesh | 40 mesh | resin, ion exchange resin, PEG, |
| | | | lactose, mannitol, |
| | | | microcrystalline cellulose, |
| | | | sorbitol, carnauba wax or other |
| | | | pharmaceutically acceptable |
| | | | carrier |
| Step 2 | 0 | 1150 | Other potent actives |
| | 0 | 1150 | Other potent actives |
| | | | Other pharmaceutically |
| | | | acceptable pH Modifiers |
| | | | Organic solvents |
| Step 3 | | | Pharmaceutically acceptable |
| | | | polymer coatings, PEG 4000 or |
| | | | higher molecular weight PEG's |
| Step 4 | | | |
| Step 5 | | | Pharmaceutically acceptable |
| | | | tableting excipients |
| Step 6 | | | Pharmaceutically acceptable |
| | | | tableting excipients |

TABLE 5
Loading and Coating Steps, T3

| | Sizing of microparticle | | | |
|---|---|---|---|---|
| Step 1 | Item | grams | Size exclusion sieving | Yield grams |
| | IRP-64 resinate | 250000 | | 11500 |
| | Drug loading of sized Microparticle | | | |
| Step 2 | Item | grams | | |
| | Sized IRP-64 | 11500 | | |
| | Liothyronine Sodium | 116.66 | | |
| | NaOH | 68 | Removed in processing | |
| | USP purified water | 101000 | Removed in processing | |
| | | | Dry weight | 11625 |
| | Making of XR-coating solution | | | |
| Step 3 | Item | grams | | |
| | Ethyl alcohol | 19440 | | |
| | USP purified water | 2160 | | |
| | Triethylcitrate | 176 | | |
| | Ethylcellulose | 2000 | | |
| | Hydroxypropyl Cellulose | 224 | | |
| | | | Dry weight | 24000 |
| | Coating of Microparticle | | | |
| Step 4 | Item | grams | | |
| | Drug loaded sized particle | 12000 | | |
| | XR-Coating solution exceeding | 6250-24000 | | |
| | 5:1 (coating solids:drug) | | | |
| | | | Total | 33600 |
| | | | Dry weight | 14160 |
| | Wet granulation of drug loaded uncoated and | | | |
| | coated microparticles | | | |
| Step 5 | Item | grams | | |
| | IR-drug loaded uncoated MP | 1060 | | |
| | XR-drug loaded coated MP | 7085 | | |
| | Mannitol | 28360 | | |
| | Microcrystalline cellulose | 9750 | | |
| | Povidone | 3750 | | |
| | USP Purified water | 13450 | Removed in processing | |
| | | | Total | 63455 |
| | | | Dry weight | 50005 |
| | Blending of granulation with tableting excipients | | | |
| Step 6 | Item | grams | | |
| | IR/XR granulation | 50000 | | |
| | Microcrystalline cellulose | 31180 | | |
| | Mannitol | 92200 | | |
| | Avicel CE15 | 44800 | | |
| | Flavor | 2240 | | |
| | lake Dye | 224 | | |
| | Mag Stearate | 3360 | | |
| | | | Total | 224004 |

TABLE 6
Basic Parameters

| Step 1 | Critical parameters | | | Notes |
|---|---|---|---|---|
| | Particle size | | | Fraction collected |
| | 80 mesh-270 | | | retained on 270 mesh |
| | mesh | | | |
| Step 2 | pH | Temp | Time | lower temp loading |
| | .02N NaOH | <60 F. | <5 hrs | for short time under |
| | solution | | | basic conditions |
| Step 3 | solubility | mixing time | | |
| | complete | >10 min | | |
| Step 4 | agglomeration | | | fluidized and sprayed |
| | <10% | | | to prevent |
| | | | | agglomeration |
| Step 5 | time | RPM | | |
| | 1-25 min | 10-500 | | |
| Step 6 | Revolutions | | | |
| | 10-200 | | | |

TABLE 7
Additional parameters

| | Ranges | | Substitutable ingredients |
|---|---|---|---|
| | Min | Max | micro particle |
| Step 1 | 400 mesh | 40 mesh | resin, ion exchange resin, PEG, |
| | | | lactose, mannitol, |
| | | | microcrystalline cellulose, |
| | | | sorbitol, carnauba wax or other |
| | | | pharmaceutically acceptable |
| | | | carrier |
| Step 2 | 0 | 1150 | Other potent actives |
| | 0 | 1150 | Other potent actives |
| | | | Other pharmaceutically |
| | | | acceptable pH Modifiers |
| | | | Organic solvents |
| Step 3 | | | Pharmaceutically acceptable |
| | | | polymer coatings, PEG 4000 or |
| | | | higher molecular weight PEG's |
| Step 4 | | | |
| Step 5 | | | Pharmaceutically acceptable |
| | | | tableting excipients |
| Step 6 | | | Pharmaceutically acceptable |
| | | | tableting excipients |

In one aspect, the present invention includes a tablet comprising, consisting essentially of, or consisting of: one or more thyroid hormones in or on a micro-multi-particulate having a mean particle size of 150 uM (micrometers) or smaller and a Span (D90−D10)/(D50) of less than 2.0, wherein the total thyroid hormone(s) are less than 10% weight-to-weight (w/w) of the micro-multi-particulate(s); and a polymer release coating on the micro-multi-particulates that is greater than a 5:1 ratio w/w, on a dry weight basis, to a dry weight of the thyroid hormone(s), wherein a total tablet weight exceeds a micro-multi-particulate weight by a ratio of 5:1, 6:1, 7:1, 8:1, 9:1, 10:1, 11:1, 12:1, 13:1, 14:1, 15:1 or greater. In one aspect, the micro-multi-particulate tablet is an extended-release chewable tablet. In another aspect, the tablet is flavored. In another aspect, the tablet has a non-gritty mouth feel, defined herein as having a bite and mouth feeling that the composition is not granular or granulated. In another aspect, the micro-multi-particulate(s) comprise a resin, ion exchange resin, PEG, lactose, mannitol, microcrystalline cellulose, sorbitol, carnauba wax or other pharmaceutically acceptable carrier. In another aspect, a mean particle size of the micro-multi-particulates is 75, 85, 95, 100, 110, 120, 130, or 140 uM, each +/−10 uM, which is the micro-multi-particulate of the present invention. In another aspect, the one or more thyroid hormones are T3, T4, or both. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulates weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In one aspect, the present invention includes a composition comprising: micro-multi-particulate(s) having a mean particle size of less than 150 uM and a Span (D90−D10)/(D50) of less than 2.0, wherein the micro-multi-particulate(s) is/are loaded with one or more thyroid hormones and the total thyroid hormone(s) are less than 10% w/w of a total micro-multi-particulate weight; and a polymer release coating on the micro-multi-particulate(s) that is greater than a 5:1 ratio w/w, on a dry weight basis, to a dry weight of total thyroid hormones; wherein the total tablet weight exceeds the micro-multi-particulate weight by a ratio of 5:1, 6:1, 7:1, 8:1, 9:1, 10:1, 11:1, 12:1, 13:1, 14:1, 15:1 or greater and is flavored. In another aspect, the composition is an extended-release chewable tablet. In another aspect, the tablet is flavored. In another aspect, the tablet has a non-gritty mouth feel. In another aspect, the micro-multi-particulate(s) comprise a resin, ion exchange resin, PEG, lactose, mannitol, microcrystalline cellulose, sorbitol, carnauba wax or other pharmaceutically acceptable carrier. In another aspect, a mean particle size of the micro-multi-particulates is 75, 85, 95, 100, 110, 120, 130, or 140 uM, each +/−10 uM. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In another aspect, the present invention includes an extended release chewable composition comprising, consisting essentially of, or consisting of: a liothyronine (T3) in or on a micro-multi-particulate having a mean particle size of less than 150 uM and a Span (D90−D10)/(D50) of less than 2.0, wherein the T3 is less than 10% w/w of a total micro-multi-particulate weight after loading; and a polymer release coating on the micro-multi-particulate(s) that is greater than a 5:1 ratio w/w, on dry weight basis to a dry weight of T3; wherein the total composition weight exceeds the micro-multi-particulate weight by a ratio of 5:1, 6:1, 7:1, 8:1, 9:1, 10:1, 11:1, 12:1, 13:1, 14:1, 15:1 or greater, and the chewable composition is flavored. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In another aspect, the present invention includes a method of making a pharmaceutical composition comprising, consisting essentially of, or consisting of: contacting one or more thyroid hormones to a micro-multi-particulate with a mean particle size of 150 uM or smaller and a Span (D90−D10)/(D50) of less than 2.0, wherein the total weight of the thyroid hormones is less than 10% of the micro-multi-particulate; and coating a polymer release on the micro-multi-particulate that is greater than a 5:1, 6:1, 7:1, 8:1, 9:1, 10:1, 11:1, 12:1, 13:1, 14:1, 15:1 ratio w/w, on a dry weight basis, to a dry weight of the thyroid hormone(s), wherein a total composition weight exceeds the micro-multi-particulate weight by a ratio of 5:1 or greater. In one aspect, the composition is an extended-release chewable tablet. In another aspect, the tablet is flavored. In another aspect, the tablet has a non-gritty mouth feel. In another aspect, the micro-multi-particulate(s) comprise a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In one aspect, the present invention includes a method of treating hypothyroidism, the method comprising, consisting essentially of, or consisting of: administering a composition comprising one or more thyroid hormones in or on a micro-multi-particulate having a mean particle size of 150 uM or smaller and a Span (D90−D10)/(D50) of less than 2.0, wherein the total thyroid hormone(s) are less than 10% weight-to-weight (w/w)(e.g., 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10%) of the micro-multi-particulate, and a polymer release coating on the micro-multi-particulate that is greater than a 5:1 ratio w/w, on a dry weight basis, to a dry weight of the thyroid hormone(s), wherein a total composition weight exceeds a micro-multi-particulate weight by a ratio of 5:1, 6:1, 7:1, 8:1, 9:1, 10:1, 11:1, 12:1, 13:1, 14:1, 15:1 or greater, in an amount sufficient to treat the hypothyroidism. In another aspect, the composition is an extended-release chewable tablet. In another aspect, the tablet is flavored. In another aspect, the tablet has a non-gritty mouth feel. In another aspect, the micro-multi-particulate(s) comprise a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the one or more thyroid hormones are T3, T4, or both. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

In another embodiment, the present invention includes a multi-particulate tablet comprising: a micro-multi-particulate comprising, consisting essentially of, or consisting of: one or more thyroid hormones, wherein the micro-multi-particulate has a mean particle size of 150 uM or smaller and a Span (D90−D10)/(D50) of less than 2.0, wherein the total thyroid hormone(s) is less than 10% weight-to-weight (w/w) of the micro-multi-particulate; and a polymer release coating on the micro-multi-particulates that is greater than a 5:1 ratio w/w, on a dry weight basis, to a dry weight of the thyroid hormone(s), wherein a total tablet weight exceeds a micro-multi-particulates weight by a ratio of 5:1 or greater. In one aspect, a dosage form is an extended-release chewable tablet. In another aspect, the tablet is flavored. In another aspect, the tablet has a non-gritty mouth feel. In another aspect, the micro-multi-particulate comprises a resin, ion exchange resin, PEG, lactose, mannitol, microcrystalline cellulose, sorbitol, carnauba wax or other pharmaceutically acceptable carrier. In another aspect, a mean particle size of the micro-multi-particulate is 75, 85, 95, 100, 110, 120, 130, or 140 uM, each +/−10 uM. In another aspect, the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight. In another aspect, the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyro acetic acid. In another aspect, a therapeutic performance of a thyroid hormone dosage of the composition or tablet meets or exceeds FDA standards for hypothyroidism, selected from at least one of: content uniformity, assay, dissolution, or stability. In one aspect, the micro-multi-particulate does not comprise a humectant. In one aspect, an immediate release portion is coated or not coated.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skilled in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those skilled in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A composition comprising:
one or more thyroid hormones in or on a micro-multi-particulate having a mean particle size of 150 uM or smaller and a Span (D90−D10)/(D50) of less than 2.0, wherein the total thyroid hormone(s) are less than 10% weight-to-weight (w/w) of the micro-multi-particulate; and
a polymer release coating on the micro-multi-particulate that is greater than a 5:1 ratio w/w, on a dry weight basis, to a dry weight of the thyroid hormone(s), wherein a total tablet weight exceeds the micro-multi-particulate weight by a ratio of 5:1 or greater.

2. The composition of claim 1, wherein the composition is a tablet, and the tablet is at least one of: an extended-release chewable tablet, a flavored tablet, or a tablet with a non-gritty mouth feel.

3. The composition of claim 1, wherein the micro-multi-particulate comprises a resin, ion exchange resin, PEG, lactose, mannitol, microcrystalline cellulose, sorbitol, carnauba wax, and optionally other pharmaceutically acceptable carrier.

4. The composition of claim 1, wherein a mean particle size of the micro-multi-particulate is 75, 85, 95, 100, 110, 120, 130, or 140 uM, each +/−10 uM.

5. The composition of claim 1, wherein the one or more thyroid hormones are T3, T4, or both.

6. The composition of claim 1, wherein the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight.

7. The composition of claim 1, wherein the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid.

8. The composition of claim 1, wherein a therapeutic performance of a thyroid hormone dosage of the composition or tablet meets or exceeds FDA standards for hypothyroidism, selected from at least one of: content uniformity, assay, dissolution, or stability.

9. The composition of claim 1, wherein the micro-multi-particulate does not comprise a humectant.

10. The composition of claim 1, wherein an immediate release portion is coated or not coated.

11. The composition of claim 1, wherein the tablet is an extended-release chewable tablet comprising:
a liothyronine (T3) in or on a micro-multi-particulate having a mean particle size of less than 150 uM and a Span (D90−D10)/(D50) of less than 2.0, wherein the T3 is less than 10% w/w of a total micro-multi-particulate weight after loading; and
a polymer release coating on the micro-multi-particulate that is greater than a 5:1 ratio w/w, on dry weight basis to a dry weight of T3;
wherein the total chewable tablet weight exceeds the micro-multi-particulate weight by a ratio of 5:1 or greater, and the chewable tablet is flavored.

12. A method of making a pharmaceutical composition comprising:
contacting one or more thyroid hormones to a micro-multi-particulate with a mean particle size of 150 uM or smaller and a Span (D90−D10)/(D50) of less than 2.0, wherein the total weight of the thyroid hormones is less than 10% of the micro-multi-particulate; and
coating a polymer release coating on the micro-multi-particulate that is greater than a 5:1 ratio w/w, on a dry weight basis, to a dry weight of the thyroid hormone(s), wherein the total pharmaceutical composition weight exceeds a micro-multi-particulate weight by a ratio of 5:1 or greater.

13. The method of claim 12, wherein the pharmaceutical composition is a table, and the tablet is at least one of: an extended-release chewable tablet, a flavored tablet, or a tablet with a non-gritty mouth feel.

14. The method of claim 12, wherein the micro-multi-particulate comprises a resin, ion exchange resin, PEG, lactose, mannitol, microcrystalline cellulose, sorbitol, carnauba wax or other pharmaceutically acceptable carrier.

15. The method of claim 12, wherein a mean particle size of the micro-multi-particulate is 75, 85, 95, 100, 110, 120, 130, or 140 uM, each +/−10 uM.

16. The method of claim 12, wherein the one or more thyroid hormones are T3, T4, or both.

17. The method of claim 12, wherein the one or more thyroid hormones are 0.02, 0.04, 0.06, 0.08, 0.09, 0.1, 0.2, 0.3, 0.4, 0.5, 0.6, 0.7, 0.8, 0.9, 1, 2, 3, 4, 5, 6, 7, 8, 9 or 10% w/w of the total micro-multi-particulate weight.

18. The method of claim 12, wherein the one or more thyroid hormones are selected from at least one of levothyroxine (T4), liothyronine (T3), N-Methyl T4 or T3, N-Ethyl T4 or T3, N-Triphenyl T4 or T3, N-Propyl T4 or T3, N-Isopropyl T4 or T3, N-Tertiary butyl T4 or T3, Sobetirome, 3,5-diiodothyropropionic acid, tetraiodothyroacetic acid, or triiodothyroacetic acid.

19. The method of claim 12, wherein a therapeutic performance of a thyroid hormone dosage of the composition or tablet meets or exceeds FDA standards for hypothyroidism, selected from at least one of: content uniformity, assay, dissolution, or stability.

20. The method of claim 12, wherein the micro-multi-particulate does not comprise a humectant.

21. The method of claim 12, wherein an immediate release portion is coated or not coated.

22. A method of treating hypothyroidism, the method comprising:
administering to a subject in need thereof a composition comprising one or more thyroid hormones in or on a micro-multi-particulate having a mean particle size of 150 uM or smaller and a Span (D90−D10)/(D50) of less than 2.0, wherein the total thyroid hormone(s) are less than 10% weight-to-weight (w/w) of the micro-multi-particulate, and a polymer release coating on the micro-multi-particulate that is greater than a 5:1 ratio w/w, on a dry weight basis, to a dry weight of the thyroid hormone(s), wherein a total composition weight exceeds a micro-multi-particulate weight by a ratio of 5:1 or greater, in an amount sufficient to treat the hypothyroidism.
